(19) United States
(12) Patent Application Publication
(10) Pub. No.: US 2013/0052696 A1
(43) Pub. Date: Feb. 28, 2013

(54) METHOD FOR PROCESSING LIGNOCELLULOSE BASED BIOMASS

(71) Applicants: Masaki Ueyama, Saitama (JP); Makoto Uda, Saitama (JP); Tsuyoshi Baba, Saitama (JP); Shoji Isobe, Saitama (JP)

(72) Inventors: Masaki Ueyama, Saitama (JP); Makoto Uda, Saitama (JP); Tsuyoshi Baba, Saitama (JP); Shoji Isobe, Saitama (JP)

(73) Assignee: HONDA MOTOR CO., LTD., Tokyo (JP)

(21) Appl. No.: 13/643,954

(22) PCT Filed: May 2, 2011
(86) PCT No.: PCT/JP2011/060518

§ 371 Date: Oct. 26, 2012

(30) Foreign Application Priority Data
May 12, 2010 (JP) 2010-110544

(51) Int. Cl. C12P 19/14

(52) U.S. Cl. 435/99

(57) ABSTRACT

A method for processing lignocellulose-based biomass capable of yielding a saccharide solution with an adequately high concentration by carrying out an enzymatic saccharification reaction of a pre-treated lignocellulose-based biomass is provided.

[Solution] The method comprises a pre-treatment step for pre-treating lignocellulose-based biomass in a reaction vessel 2 to dissociate lignin from the lignocellulose-based biomass, or swell the lignocellulose-based biomass, to yield a first processed product; a first saccharification treatment step for carrying out partially an enzymatic saccharification reaction in a reaction vessel 3 of the first processed product to yield a second flowable processed product; a transfer step for transferring the second processed product to a reaction vessel 5 without contact with the outside air; and a second saccharification treatment step for carrying out an enzymatic saccharification reaction in the reaction vessel 5 of the second processed product to yield a saccharide solution.

### TECHNICAL FIELD

The present invention relates to a method for processing lignocellulose-based biomass.

### BACKGROUND ART

From a viewpoint of prevention of global warming, reduction of the volume of carbon dioxide emission which is believed to be one of the causes thereof has been required recently. To this end, use of a blend fuel of a liquid hydrocarbon such as gasoline and ethanol for an automobile fuel has been studied.

The ethanol used here can be produced by fermentation of plant substances, e.g. farm products, such as sugarcane and corn. Since plants themselves, which are source materials of the plant substances, have absorbed carbon dioxide by photosynthesis, when ethanol derived from the plant substances are burned, the amount of emitted carbon dioxide is equal to the amount of the carbon dioxide having been absorbed by the plants themselves. In other words, the so-called carbon-neutral effect can be obtained, such that the overall emission amount of carbon dioxide becomes zero in theory.

On the other hand, there is a drawback that large scale consumption of the sugarcane or corn as a source material for ethanol would reduce the amount of food supply.

Consequently, a technique for producing ethanol using nonfood lignocellulose-based biomass as the plant substances instead of sugarcane, corn, etc. has been studied. Since the lignocellulose-based biomass contains cellulose, ethanol can be yielded by degrading the cellulose by enzymatic saccharification to a saccharide such as glucose, and fermenting the product saccharide.

Examples of the lignocellulose-based biomass include wood, rice straw, wheat straw, bagasse, bamboo, a stem, leaf and cob of corn, pulp, and a waste therefrom such as wastepaper.

Meanwhile, since the lignocellulose-based biomass includes as major constituents hemicellulose and lignin in addition to cellulose, and the cellulose and the hemicellulose are normally bound tightly to the lignin, an enzymatic saccharification reaction with the cellulose is inhibited as it is. Consequently, for an enzymatic saccharification reaction of the lignocellulose as a substrate it is desirable to dissociate lignin from the substrate in advance, or have the substrate swollen, so that the enzyme should be able to contact with the substrate.

In this regard, the term “dissociate” means herein at least a part of the bonds between lignin and cellulose or hemicellulose is broken. The term “swell” means crystalline cellulose expands due to infiltration of a liquid, which generates gaps in cellulose or hemicellulose constituting the crystalline cellulose, or gaps inside a cellulose fiber.

Consequently, when ethanol is produced from the lignocellulose-based biomass, the lignocellulose-based biomass is crushed and pre-treated to dissociate lignin from the lignocellulose-based biomass, or to have the lignocellulose-based biomass swollen. Then the pre-treated lignocellulose-based biomass is subjected to an enzymatic saccharification reaction to yield a saccharide solution, which is then fermented to produce ethanol.

The pre-treatment and the enzymatic saccharification reaction are generally conducted in separate reaction vessels (e.g. see Patent Literature 1). In this connection, when crushed lignocellulose-based biomass is pre-treated as above, the lignocellulose-based biomass becomes a powder in wet state with very low flowability, and requires enormous energy for agitation, if a large amount thereof is processed at once. Therefore, by carrying out the pre-treatment of lignocellulose-based biomass in a relatively small-sized reaction vessel, and transferring the pre-treated lignocellulose-based biomass to another reaction vessel for carrying out an enzymatic saccharification reaction, a batchwise treatment can be performed efficiently.

If the pre-treatment and the enzymatic saccharification reaction are conducted in separate reaction vessels as described above, since the pre-treated lignocellulose-based biomass is a powder in wet state with very low flowability as described above, its transfer through a pipeline to a reaction vessel for an enzymatic saccharification reaction is difficult. Therefore, the pre-treated lignocellulose-based biomass is usually discharged from the reaction vessel for a pre-treatment and transferred to a transportation container, etc., and transferred to the reaction vessel for an enzymatic saccharification reaction via the transportation container, etc.

### CITATION LIST

### Patent Literature

Patent Literature 1: Japanese Patent Laid-Open No. 2008-271962

### SUMMARY OF INVENTION

### Technical Problem

However, if pre-treated lignocellulose-based biomass is once transferred to a transportation container, etc., transferred to a reaction vessel for an enzymatic saccharification reaction from the transportation container, etc. and subjected to the enzymatic saccharification reaction, there is a drawback that a saccharide solution with an adequately high concentration can not be sufficiently obtained.

Consequently, in order to eliminate such a drawback, an object of the present invention is to provide a method for processing lignocellulose-based biomass capable of yielding a saccharide solution with an adequately high concentration by means of an enzymatic saccharification reaction on pre-treated lignocellulose-based biomass.

### Solution to Problem

The inventors have intensively investigated reasons for such a failure in yielding a saccharide solution with an adequately high concentration, if pre-treated lignocellulose-based biomass is once transferred to a transportation container, etc., transferred to a reaction vessel for an enzymatic saccharification reaction from the transportation container, etc. and subjected to the enzymatic saccharification reaction.

As a result, it has been found that, if pre-treated lignocellulose-based biomass is transferred via a transportation container, etc. to a reaction vessel for an enzymatic saccharification reaction, the lignocellulose-based biomass can be microbially contaminated during the transfer of the lignocellulose-based biomass to the transportation container, etc. or during the transfer to the reaction vessel. If the contaminated lignocellulose-based biomass is subjected to an enzymatic saccharification reaction, some of the product saccharide would be consumed by the microorganism to provide a saccharide solution that is not at an adequately high concentration.

Based on such findings, the inventors have deepened the investigation, thereby completing the present invention.

Therefore, the present invention provides a method for processing lignocellulose-based biomass by pre-treating lignocellulose-based biomass in a reaction vessel and then transferring the biomass to another reaction vessel for enzymatic saccharification to yield a saccharide solution, the method comprising: a pre-treatment step for pre-treating the lignocellulose-based biomass in a first reaction vessel to dissociate lignin from the lignocellulose-based biomass, or swell the lignocellulose-based biomass, to yield a first processed product; a first saccharification treatment step for carrying out partially an enzymatic saccharification reaction in a second reaction vessel of the first processed product yielded in the pre-treatment step to yield a second flowable processed product; a transfer step for transferring the second processed product yielded in the first saccharification treatment step to a third reaction vessel without contact with the outside air; and a second saccharification treatment step for carrying out an enzymatic saccharification reaction in the third reaction vessel of the second processed product transferred in the transfer step to yield a saccharide solution.

According to the method for processing lignocellulose-based biomass of the present invention, in the pre-treatment step lignocellulose-based biomass is first pre-treated in a first reaction vessel to dissociate lignin from the lignocellulose-based biomass or swell the lignocellulose-based biomass. As a result, the first processed product can be obtained, in which cellulose or hemicellulose contained in the lignocellulose-based biomass is capable of an enzymatic saccharification reaction.

The first processed product has been yielded by pre-treating the lignocellulose-based biomass, and is, for example, a powder in wet state, which is not flowable. Therefore, next as a first saccharification treatment step, the first processed product yielded in the pre-treatment step is subjected to a partial enzymatic saccharification reaction in the second reaction vessel. As a result, a part of the cellulose or hemicellulose contained in the lignocellulose-based biomass is saccharified to yield a second flowable processed product.

In the first saccharification treatment step, it is required to saccharify enzymatically the first processed product only so as to make the first processed product flowable, but not required to saccharify all the cellulose or hemicellulose contained in the lignocellulose-based biomass.

In the next transfer step, the second processed product is transferred to a third reaction vessel without contact with the outside air. Since the second processed product is flowable, it can be directly transferred from the second reaction vessel to the third reaction vessel through a pipeline by a transfer unit such as a centrifugal pump without transferring to a transportation container, etc. As a result, the second processed product does not contact with the outside air, and thus contamination of the second processed product can be prevented.

Next, in the second saccharification treatment step, the second processed product transferred in the transfer step is subjected to an enzymatic saccharification reaction in the third reaction vessel. As a result, the cellulose or hemicellulose remaining in the lignocellulose-based biomass not saccharified in the first saccharification treatment step can be saccharified to yield a saccharide solution.

Since the second processed product is kept under a condition out of contact with the outside air in the transfer step, it is not contaminated, and a saccharide yielded in the second saccharification treatment step is not consumed by the microorganism. Consequently, a saccharide solution with an adequately high concentration can be yielded in the third reaction vessel. Since the saccharide solution contains a saccharide at an adequately high concentration, it can be used favorably as a source material for producing ethanol by ethanol fermentation.

According to a method for processing lignocellulose-based biomass of the present invention, the first processed product yielded by the pre-treatment is poor in flowability, and therefore if a large amount thereof is to be processed at once, enormous energy is required for agitation. By conducting the pre-treatment, the first saccharification treatment step, and the second saccharification treatment in separate reaction vessels, the pre-treatment and the first saccharification treatment step can be conducted in a relatively small first reaction vessel and second reaction vessel respectively in small amounts, so that efficient batchwise treatments can be carried out.

Then, after the pre-treatment and the first saccharification treatment step, the second flowable processed product is subjected to the second saccharification treatment step in a third reaction vessel to yield efficiently a saccharide solution with an adequately high concentration.

Further, in the method for processing lignocellulose-based biomass of the present invention, in order to prevent contamination of the second processed product more reliably, it is also desirable to protect the first processed product yielded in the pre-treatment step from contamination. However, since the first processed product yielded by the pre-treatment is poor in flowability as described above, transfer without contact with the outside air is difficult. Consequently, in the method for processing lignocellulose-based biomass of the present invention, it is preferable that the first reaction vessel and the second reaction vessel are a common reaction vessel.

In such an event, after the pre-treatment in the first reaction vessel yielding the first processed product, using the first reaction vessel as it is also as the second reaction vessel, the first processed product can be subjected to an enzymatic saccharification reaction in the reaction vessel. As a result, the transfer of the first processed product from the first reaction vessel to the second reaction vessel is not necessary and the contamination of the first processed product can be prevented.

Further, with respect to a method for processing lignocellulose-based biomass of the present invention, an enzymatic saccharification reaction on the first processed product in the first saccharification treatment step and an enzymatic saccharification reaction on the second processed product in the second saccharification treatment step are carried out preferably using an enzyme for hydrolyzing cellulose and hemicellulose. In such a way, saccharides can be yielded from both cellulose and hemicellulose, so that the concentration of the saccharide solution can be made high.

Further, with respect to a method for processing lignocellulose-based biomass of the present invention, the second processed product has preferably the viscosity in a range of 30 to 1000 mPa·s. If the second processed product has the viscosity exceeding 1000 mPa·s, the transfer by a general-purpose transfer unit such as a centrifugal pump is difficult. Meanwhile, to provide a second processed product with the viscosity below 30 mPa·s, retention for a prolonged time period at a constant temperature is required and the production cost can hardly be lowered.

### BRIEF DESCRIPTION OF DRAWINGS

FIG. 1 is a system configuration diagram showing a configuration example of a processing system used in the method for processing lignocellulose-based biomass of the present invention.

FIG. 2 is a system configuration diagram showing another configuration example of a processing system used in the method for processing lignocellulose-based biomass of the present invention.

FIG. 3 is a perspective partially-cutaway view of a reaction vessel 7 shown in FIG. 2.

FIG. 4 is a graph showing a temporal change of the viscosity of a processed product in the first saccharification treatment step according to the present invention.

FIG. 5 is a graph showing temporal changes of glucose concentrations of saccharide solution yielded in the second saccharification treatment step according to the present invention.

### DESCRIPTION OF EMBODIMENTS

An embodiment of the present invention will be described in more detail referring to the appended drawings.

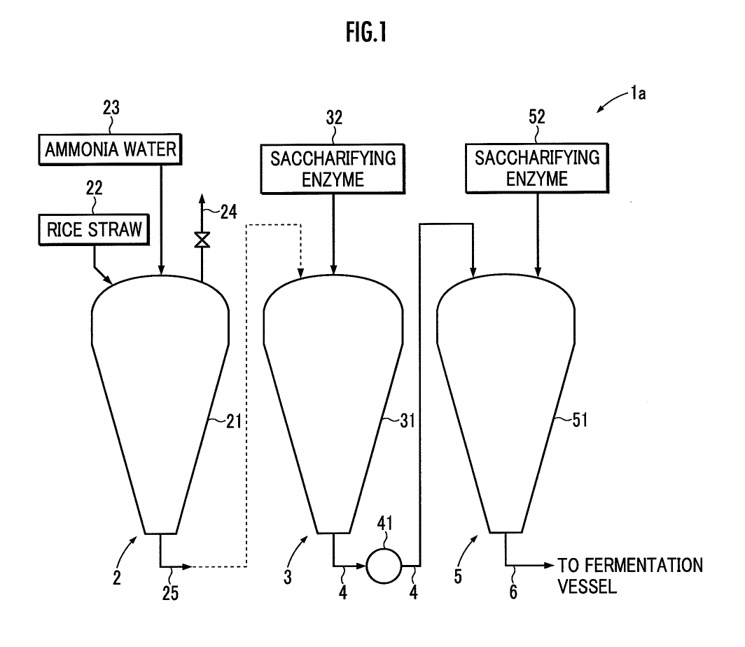

The method for processing lignocellulose-based biomass in this embodiment uses rice straw as lignocellulose-based biomass and can be carried out, for example, by a processing system 1a shown in FIG. 1.

The processing system 1a comprises a first reaction vessel 2, a second reaction vessel 3, a first line 4 originating from the bottom of the reaction vessel 3, a third reaction vessel 5 connected with the line 4, and a second line 6 originating from the bottom of the reaction vessel 5.

The first reaction vessel 2 comprises a reactor 21 accommodating rice straw for yielding a first processed product by pre-treating the rice straw as lignocellulose-based biomass. The reaction vessel 2 further comprises a rice straw feed port 22, an ammonia water feed port 23, and a pressure regulation port 24 on the top of the reactor 21, and a discharge port 25 for discharging the first processed product at the bottom.

The second reaction vessel 3 comprises a reactor 31 containing the first processed product for carrying out partially an enzymatic saccharification treatment of the first processed product to yield a second flowable processed product. The reaction vessel 3 further comprises an enzyme feed port 32 on the top of the reactor 31.

The first line 4 is a transfer unit which transfers the second flowable processed product yielded in the reaction vessel 3 to the third reaction vessel 5 without contact with the outside air, and comprises a pump 41 halfway. As the pump 41, a centrifugal pump, a Moineau pump, etc. can be applied.

The third reaction vessel 5 comprises a reactor 51 containing the second processed product for carrying out an enzymatic saccharification treatment of the second processed product transferred through the line 4 to yield a saccharide solution. The reaction vessel 5 further comprises an enzyme feed port 52 on the upper portion of the reactor 51.

The second line 6 is a transfer unit which transfers the saccharide solution yielded in the third reaction vessel 5 to a fermentation vessel (not illustrated) in the next step.

Next, the method for processing lignocellulose-based biomass in this embodiment using the processing system 1a shown in FIG. 1 will be described.

In the method for processing lignocellulose-based biomass in this embodiment, firstly a pre-treatment step is carried out in the first reaction vessel 2. In the pre-treatment step, firstly rice straw, which is lignocellulose-based biomass, is charged as a substrate through the rice straw feed port 22 into the reactor 21. To facilitate the charging, the rice straw has been chopped, for example, by a cutter mill to a size allowing it to pass through a 3-mm mesh opening.

Next, ammonia water is charged through the ammonia water feed port 23, while the rice straw is agitated in the reactor 21, to obtain a substrate mixture of the rice straw and the ammonia water. Next, the substrate mixture is heated in the reactor 21 and maintained at a predetermined temperature for a predetermined time period to dissociate lignin from the rice straw or to swell the rice straw to yield the first processed product.

Then, the first processed product is further heated in the reactor 21 to evaporate ammonia. The evaporated ammonia is emitted from the reactor 21 through, for example, the pressure regulation port 24.

The resulting first processed product is a powder in wet state, and is lacking flowability. Consequently, the first processed product is discharged from the discharge port 25 provided at the bottom portion of the first reaction vessel 2 and transferred to a transportation container, etc. not illustrated. Then the same is supplied by the transportation container, etc. as indicated by a broken line in FIG. 1, to the reactor 31 of the second reaction vessel 3.

Then, the first saccharification treatment step is carried out in the second reaction vessel 3. In the first saccharification treatment step, firstly, a pH adjuster is added through a pH adjuster feed port not illustrated to the reactor 31 to adjust the pH of the first processed product yielded in the pre-treatment step to a desired pH, for example, pH in a range of 4 to 4.5. As the pH adjuster, an acid such as dilute sulfuric acid can be used.

Next, saccharifying enzymes for degrading cellulose and hemicellulose are charged through the enzyme feed port 32 into the reactor 31 at a predetermined ratio, and according to need water may be further added to provide a desired water content. As the saccharifying enzymes for degrading cellulose and hemicellulose, cellulase, hemicellulase, etc. can be used.

Specific examples of the saccharifying enzymes include GC220, Accellerase 1000 and Accellerase XC, Accellerase 1000 and Accellerase XY, Accellerase 1500 and Accellerase XC, Accellerase 1500 and Accellerase XY (the above are made by Genencor Inc.), Meicelase®, Acremonium cellulase (the above are made by Meiji Seika Co., Ltd.), and Cellic CTec and Cellic HTec (made by Novozymes A/S).

Next, the temperature in the reactor 31 is adjusted for carrying out an enzymatic saccharification reaction by the degrading enzymes of the first processed product. As a result, a part of cellulose and hemicellulose contained in the rice straw in the first processed product is hydrolyzed to produce a saccharide and yield a second flowable processed product.

The second processed product is in a slurry state or a liquid state, and has the viscosity in a range of 30 to 1000 mPa·s. As a result, the second processed product can be transferred as a fluid through the line 4 by the pump 41, such as a centrifugal pump or a Moineau pump.

In the first saccharification treatment step, it is required that only a part of cellulose and hemicellulose contained in the rice straw in the first processed product should be saccharified to make it flowable, but not that all the cellulose and hemicellulose should be saccharified. Therefore, a decision to terminate the first saccharification treatment step is made based on any of the following methodologies.

The first methodology is to confirm visually that the first processed product has been modified to a slurry or viscous liquid state.

The second methodology is, after the pre-treatment step is finished and the first saccharification treatment step is started, to extract samples from time to time to measure the viscosities thereof, and terminate the first saccharification treatment step, when the viscosity of one of them has reached a predetermined value.

The third methodology is to fix values of parameters concerning the first saccharification treatment, such as temperature, and agitation speed and initiate the saccharification treatment of the first saccharification treatment step, and terminate the saccharification treatment of the first saccharification treatment step after an elapse of a time period which should be necessary for the first processed product to reach a predetermined viscosity. The time period necessary for the first processed product to reach a predetermined viscosity may be determined in advance, by changing the values of the parameters and measuring the time required from the initiation of the first saccharification treatment step until the first processed product reaches a predetermined viscosity. Meanwhile, in the current embodiment, the third methodology is employed to decide the termination of the first saccharification treatment step.

When the first saccharification treatment step is terminated, and the second processed product is yielded, then a transfer step is carried out. In the transfer step, the second processed product is taken out from the bottom of the second reaction vessel 3 through the line 4, and the second processed product is transferred by the pump 41, such as a centrifugal pump or a Moineau pump, through the line 4 to the third reaction vessel 5. Since the transfer is carried out through the line 4, the second processed product can be transferred to the reaction vessel 5 without contact with the outside air.

Then, when the second processed product is transferred to the reaction vessel 5, the second saccharification treatment step is carried out in the reaction vessel 5. In the second saccharification treatment step, the temperature in the reactor 51 is adjusted for carrying out an enzymatic saccharification reaction by the saccharifying enzyme of the second processed product. In this regard, as the saccharifying enzymes, the saccharifying enzymes charged into the reactor 31 and carried over from the first processed product to the second processed product may be used as they are contained therein, or additional saccharifying enzymes may be charged through the enzyme feed port 52 into the reactor 51. As the saccharifying enzymes charged through the enzyme feed port 52 may be the same saccharifying enzymes as used for the first saccharification treatment.

As a result, cellulose and hemicellulose contained in the second processed product are hydrolyzed to produce a saccharide. The cellulose and hemicellulose contained in the second processed product is a remaining portion of the cellulose or hemicellulose contained in the lignocellulose-based biomass, which has not been saccharified in the first saccharification treatment step.

Since the second processed product has been transferred to the reaction vessel 5 through the line 4 without contact with the outside air, it is not contaminated, and therefore a saccharide yielded in the second saccharification treatment step is not consumed by the microorganism. Consequently, in the second saccharification treatment step a saccharide solution with an adequately high concentration can be obtained in the reaction vessel 5.

The saccharide solution is transferred through the line 6 to a fermentation vessel not illustrated after the completion of the second saccharification treatment step. Since the saccharide solution contains a saccharide at an adequately high concentration, it can be used favorably as a source material for producing ethanol by ethanol fermentation.

In the current embodiment, the pre-treatment step and the first saccharification treatment step are carried out using separate reaction vessels 2 and 3. However, the first processed product yielded in the pre-treatment step is, as described above, a powder in wet state and not flowable. There is a concern, therefore, if the first processed product is transferred from the first reaction vessel 2 to the second reaction vessel 3 using a transportation container, etc., that the first processed product may contact with the outside air and be contaminated during the transfer.

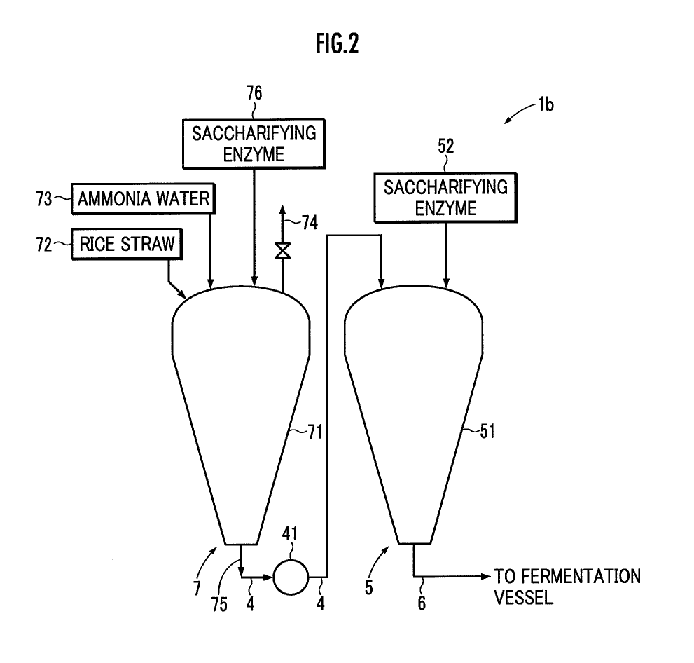

Therefore, the method for processing lignocellulose-based biomass is this embodiment is preferably carried out using a processing system 1b, in which the first reaction vessel and the second reaction vessel are combined to a common reaction vessel 7 as shown in FIG. 2.

The processing system 1b has exactly the same configuration as the processing system 1a shown in FIG. 1, except that instead of the first reaction vessel 2 and the second reaction vessel 3, the reaction vessel 7 combining the first reaction vessel and the second reaction vessel 3 is provided. In other words, the processing system 1b comprises a first line 4 originating from the bottom of the reaction vessel 7, a third reaction vessel 5 connected with the line 4, and a second line 6 originating from the bottom of the reaction vessel 5.

The reaction vessel 7 comprises a reactor 71 accommodating rice straw as the lignocellulose-based biomass and a first processed product in order to pre-treat the rice straw yielding a first processed product, and to saccharify enzymatically a part of the first processed product yielding a flowable second processed product. The reaction vessel 7 further comprises a rice straw feed port 72, an ammonia water feed port 73, a pressure regulation port 74, and an enzyme feed port 75 on the upper portion of the reactor 71, and a discharge port 76 for discharging the second processed product at the bottom part.

In the method for processing lignocellulose-based biomass in this embodiment using the processing system 1b shown in FIG. 2, lignocellulose-based biomass can be processed exactly identically as in the case of the processing system 1a, except that the pre-treatment and the first saccharification treatment are carried out in a single reaction vessel 7. In the event that the processing system 1b shown in FIG. 2 is used, both the first processed product and the second processed product do not contact with the outside air, and are protected surely from contamination, therefore a saccharide solution with a higher concentration can be obtained in the reaction vessel 5.

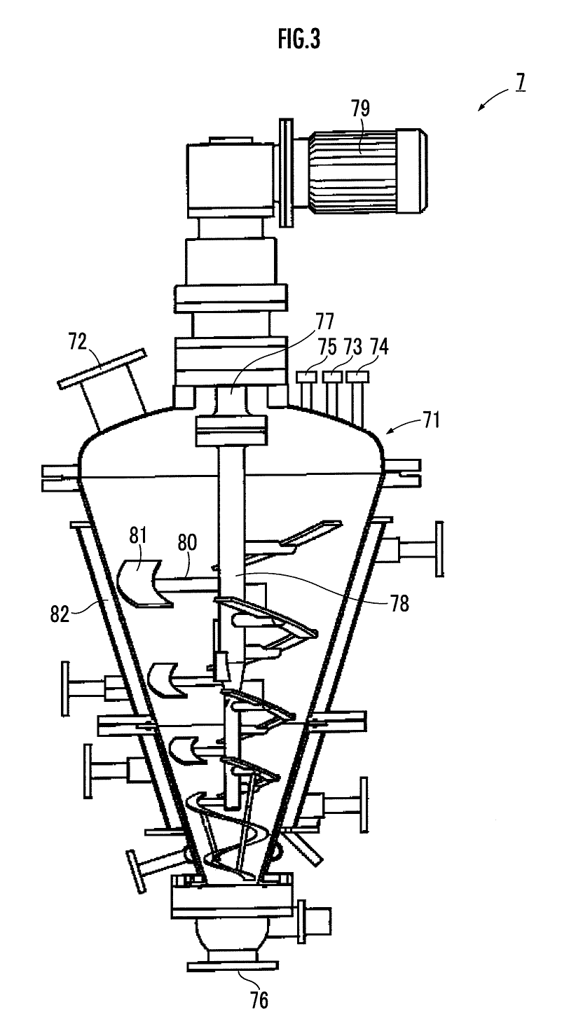

In the method for processing lignocellulose-based biomass in this embodiment, the reaction vessel 7 that is used and shown in FIG. 2 can have, for example, the configuration shown in FIG. 3.

The reaction vessel 7 shown in FIG. 3 comprises a reactor 71 formed in an inverse-conical shape for mixing the rice straw and the first processed product therein. The reaction vessel 7 comprises a rice straw feed port 72, an ammonia water feed port 73, a pressure regulation port 74, and an enzyme feed port 75, as well as a heating medium feed port, a heating medium discharge port, and a pH adjuster feed port, which are not illustrated, on the upper portion of the reactor 71, and a discharge port 76 connected with the first line 4 at the bottom part.

In the reactor 71 are provided a driving shaft 77 and a vertical shaft 78 rotatably suspended by the driving shaft 77, and the driving shaft 77 is driven to rotate by a driving unit 79 such as an electric motor placed at the upper portion of the reactor 71. Further in the reactor 71, at the tips of arms 80 extended in the horizontal direction from the vertical shaft 78 are provided agitator blades 81.

In the reaction vessel 7 the vertical shaft 78 is driven to rotate by the driving unit 79 through the driving shaft 77, so as to rotate the agitator blades 81 attached to the vertical shaft 78. By the above, rice straw charged into the reactor 71 is mixed with ammonia water, etc. and agitated, or the first processed product is mixed with a saccharifying enzyme and agitated.

Further, at the outer surface of the reactor 71 is provided a jacket 82, which is so constituted that a heating medium can circulate for regulating the temperature in the reactor 71. In the jacket 82, the heating medium introduced through the heating medium feed port circulates and flows out through the heating medium discharge port.

Further, in the pre-treatment step of this embodiment, the lignocellulose-based biomass is charged with ammonia water and maintained at a predetermined temperature for a predetermined time period. However, in the pre-treatment step, a hydrothermal treatment can also be carried out, in which water is added to the lignocellulose-based biomass so that it has a predetermined water content, the reaction vessel 2 or the reaction vessel 7 is hermetically closed, the temperature is raised by heating with agitation to a predetermined temperature and maintained for a predetermined time period, and then the temperature of the lignocellulose-based biomass is lowered by opening the pressure regulation port 24 or the pressure regulation port 74.

Next, Example of the present invention will be described.

### EXAMPLE

In the current Example, as lignocellulose-based biomass, 386 kg of air-dried rice straw with a water content of 12 mass % was chopped by a cutter mill to a size passing through a 3-mm mesh opening. Then, the rice straw was supplied to the reaction vessel 7 shown in FIG. 3 (PV Mixer with internal volume: 2000 L, made by Kobelco Eco-Solutions Co., Ltd.).

Next, with respect to the dry mass of 340 kg of the 386 kg-rice straw, 340 kg of 25 mass % ammonia water was supplied to the reaction vessel 7 with agitation to obtain a substrate mixture. In the substrate mixture, the mass ratio of the mass of the ammonia water to the dry mass of the rice straw is 1:1.

Next, as the pre-treatment step, the substrate mixture was maintained at the temperature of 80° C. for 8 hours, and then heated from the outside with agitation to evaporate ammonia to provide a water content of 7.79 mass % and a remaining ammonia concentration of 0.25 mass %, and thus the first processed product.

Next, as the first saccharification treatment step, the first processed product was adjusted by 5 mass %-dilute sulfuric acid to a pH range of 4 to 4.5, then 15 kg of a saccharifying enzyme was added, and further water was added to make the concentration of the rice straw as the lignocellulose-based biomass to be 26 mass %. Then the first processed product was agitated, while maintaining the reaction temperature at 50° C., to hydrolyze a part of cellulose and hemicellulose to produce a saccharide, thereby obtaining a second flowable processed product.

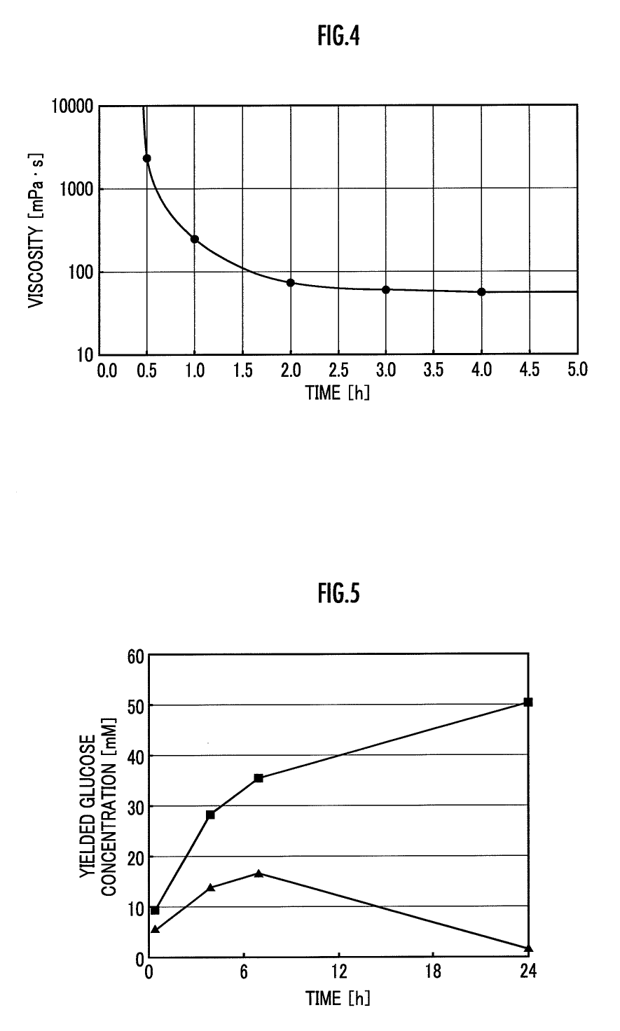

During the above, samples were taken at 0.5 hours, 1 hour, 2 hours, 3 hours, 4 hours, and 72 hours after the initiation of the first saccharification treatment step, and the viscosity of the second processed product was measured. The results are shown in FIG. 4.

As shown in FIG. 4, compared to the viscosity immediately after the initiation of the first saccharification treatment (after 0.5 hours), the viscosity 1 hour after the initiation of the saccharification decreases to about 1/10, namely to a viscosity not more than 1000 mPa·s, which is obviously in a flowable state.

The viscosity of the second processed product decreased slowly to 73.2 mPa·s (25.2° C.) after 2 hours, 61.9 mPa·s (26.7° C.) after 3 hours, 56.7 mPa·s (27.6° C.) after 4 hours, and 30.9 mPa·s (25.1° C.) after 72 hours from the initiation of the saccharification, and therefore sharp decrease in the viscosity was not observed after an elapse of 2 hours from the initiation of the saccharification. The temperature indicated after the viscosity means the liquid temperature at a measurement.

Then, the second flowable processed product yielded in the first saccharification treatment step was taken out of the reaction vessel 7 and transferred through the line 4 to the reaction vessel 5 without contact with the outside air, and was subjected to a saccharification treatment under the conditions of pH 5.5 and 50° C. in the reaction vessel 5 as the second saccharification treatment step.

Samples were taken immediately, 4 hours, 7 hours, and 24 hours after the second saccharification treatment was initiated, and the glucose concentrations were measured. The results are shown as Example in FIG. 5.

Also, a saccharification treatment was carried out exactly identically with the current Example, except that after the pre-treatment, without conducting a first saccharification treatment step, a first processed product was discharged from the reaction vessel 7 to a transportation container, and then transferred to the reaction vessel 5, and the glucose concentration was measured. The results are shown as Comparative Example in FIG. 5.

In FIG. 5, the plot with square points shows a glucose concentration change in Example, and the plot with triangle points shows a glucose concentration change in Comparative Example.

As shown in FIG. 5, in the Comparative Example, the concentration of product glucose increases initially, but then decreases with the lapse of saccharification time. This is presumably because the first processed product was contaminated during the transfer from the reaction vessel 7 to the reaction vessel 5 using the transportation container, and the product glucose was partially consumed by the microorganism. On the other hand, in the Example, compared to the Comparative Example, the glucose concentration is higher even immediately after the initiation of the saccharification, and becomes still higher with the lapse of saccharification time.

It is obvious therefore that a saccharide solution with an adequately high concentration can be yielded by transferring a first processed product and a second processed product to the reaction vessel 5 without contact with the outside air, and yielding a saccharide solution by carrying out an enzymatic saccharification reaction in the reaction vessel 5.

### REFERENCE SIGNS LIST

1: processing system, 2, 3, 5, 7: reaction vessel, 21, 71: reactor, 22, 72: rice straw feed port, 23, 73: ammonia water feed port, 24, 75: enzyme feed port, 25, 74: pressure regulation port,26, 76: discharge port, 77: driving shaft, 78: vertical shaft, 79: driving unit, 81: agitator blade, 82: jacket.

1. A method for processing lignocellulose-based biomass by pre-treating lignocellulose-based biomass in a reaction vessel and then transferring the biomass to another reaction vessel for enzymatic saccharification to yield a saccharide solution, the method comprising:
a pre-treatment step for pre-treating the lignocellulose-based biomass in a first reaction vessel to dissociate lignin from the lignocellulose-based biomass, or swell the lignocellulose-based biomass, to yield a first processed product;
a first saccharification treatment step for carrying out partially an enzymatic saccharification reaction in a second reaction vessel of the first processed product yielded in the pre-treatment step to yield a second flowable processed product;
a transfer step for transferring the second processed product yielded in the first saccharification treatment step to a third reaction vessel in a state without contact with the outside air; and
a second saccharification treatment step for carrying out an enzymatic saccharification reaction in the third reaction vessel of the second processed product transferred in the transfer step to yield a saccharide solution.

2. The method for processing lignocellulose-based biomass according to claim 1, wherein the first reaction vessel and the second reaction vessel are a common reaction vessel.

3. The method for processing lignocellulose-based biomass according to claim 1, wherein the enzymatic saccharification reaction of the first processed product in the first saccharification treatment step, and the enzymatic saccharification reaction of the second processed product in the second saccharification treatment step are carried out using an enzyme for hydrolyzing cellulose and hemicellulose.

4. The method for processing lignocellulose-based biomass according to claim 1, wherein the second processed product has a viscosity in a range of 30 to 1000 mPa·s.
